(19) United States
(12) Patent Application Publication
(10) Pub. No.: US 2008/0044363 A1
(43) Pub. Date: Feb. 21, 2008

(54) Therapeutic Dental Composition

(75) Inventor: R. Eric Montgomery, Monterey, MA (US)

Correspondence Address: DISCUS DENTAL IMPRESSIONS, INC., 8550 HIGUERA STREET, CULVER CITY, CA, 90232, US

(73) Assignee: BRITESMILE PROFESSIONAL, LLC, Culver City, CA (US)

(21) Appl. No.: 11/927,129

(22) Filed: Oct. 29, 2007

Related U.S. Application Data
(63) Continuation of application No. 10/846,207, filed on May 14, 2004, now abandoned.
(60) Provisional application No. 60/471,427, filed on May 16, 2003.

(51) Int. Cl. A61K 8/00; A61P 43/00

(52) U.S. Cl. 424/49

(57) ABSTRACT

A solid or semi-solid oral therapeutic dental composition comprising a therapeutic agent dispersed in a water-insoluble or water-resistant carrier. The dental composition may be in the form of a crayon or dispensed from a delivery device such as a pen or the like.

### RELATED APPLICATIONS

This application claims priority to U.S. Provisional Application No. 60/471,426, filed May 16, 2003, which is incorporated by reference herein in its entirety.

### BACKGROUND OF THE INVENTION

For years, dental compositions such as tooth whiteners and fluoride gels have been applied to the teeth of patients in need of a cosmetic or therapeutic dental treatment. These compositions, usually in the form of a gel, paste or foam, have traditionally been comprised of one or more active therapeutic agents dissolved in an aqueous or water-soluble carrier. An aqueous or water-soluble carrier was used because the majority of dental therapeutic agents, such as inorganic fluorides (anticaries agents), peroxides (tooth whitening agents), chlorhexidine (an antibacterial agent), potassium nitrate (a tooth desensitizing agent) and polyphosphates (tartar control agents) are water-soluble compounds. Most of these compositions are intended to be brushed, rinsed or sprayed onto the teeth for a short period of time, for instance the few minutes during which the average individual will brush his or her teeth.

A number of dental treatments, though, require that a therapeutic agent be in contact for a much longer period of time than is practical by the above methods. Arch-shaped dental trays have been developed that are either customized by methods known in the art, or are in the one-size-fits-all category. Such trays are typically loaded with a small amount of a dental composition, placed over the teeth of the upper arch, the lower arch, or both simultaneously (using two dental trays at the same time). While such devices and methods tend to greatly increase the length of time a dental composition remains in contact with the tooth surfaces, said compositions still remain highly soluble in water. Being soluble in water, these compositions rapidly dissolve and leach out of the dental tray once placed in the mouth in contact with saliva. Upon dilution, prior art compositions are seen to quickly migrate out of the dental tray and thus become free to contact areas of the oral cavity not intended for treatment. The diluted compositions may also be swallowed by the patient, for instance when a tooth whitening composition is loaded into a dental tray and placed in the oral cavity against the teeth for an extended period of time, such as overnight while the patient is asleep. The whitening composition within the tray gradually becomes lower in viscosity due to dilution with saliva, migrates out of the dental tray, and is unintentionally swallowed by the patient. Repeated ingestion of the oxidizing ingredients of such compositions may be detrimental to the tissue surfaces of the digestive tract.

Other delivery modes for applying tooth whitening compositions to the surfaces of the teeth have recently become available, each with different degrees of acceptance by the consumer and effectiveness. Crest Whitestrips (Procter & Gamble, Cincinnati, Ohio) are thin plastic strips that are coated on one side with a layer of tooth whitening gel and stored until use against a release backing (similar to a pressure sensitive label), in a unit dose laminated pouch. When a consumer or patient desires tooth whitening, the pouch is opened, the strip/gel combination is removed from the release backing, and the user carefully places the strip on the teeth to be whitened (gel side against the teeth). While this approach is safe and effective, the strips are somewhat cumbersome to handle and position, and there are instances where the entire plastic strip and associated gel have been swallowed by the consumer. The Crest Whitestrips gel is also water soluble, so salivary intrusion and gel dissolution occurs in a similar fashion to that observed with dental whitening trays.

Crest® Night Effects™ is a gel tooth whitening system containing 19% sodium percarbonate that is used at night to remove tooth stains. The tooth whitening gel is brushed onto the teeth and then forms a film coating that releases the sodium percarbonate. The gel includes volatile ingredients, such as ethyl acetate, that are used to thin the gel before use, but once on the teeth, “flash” off and the composition thickens to a film. As a result, Crest® Night Effects™ can be cumbersome to use as it is recommended that the mouth remain open for at least 30 seconds to help the film coating set.

Simply White (Colgate, Piscataway, N.J.) is a brush-on tooth whitening gel that is simply brushed onto the teeth using a nail polish type applicator bottle and brush combination. The brush handle tip is dipped into the bottle reservoir, picking up an amount of gel that is transferred to the teeth by dabbing or brushing the surfaces. The gel, which contains ethyl alcohol, is allowed to dry for 30 seconds before the user can close his or her mouth. This period of time is necessary for the gel to dry and become sufficiently thick so as not to immediately wash from the tooth surfaces due to salivary flow. Even so, the residence time of the Simply White gel is quite short, for example approximately 3 to 7 minutes. This period of time is insufficient for penetration of peroxide into the tooth to effectuate whitening, as is evidenced by the poor performance of this product.

### SUMMARY OF INVENTION

The present invention relates to a dental composition having a therapeutic agent dispersed in a water-insoluble or water-resistant carrier, such that when placed in the oral cavity, the composition does not dissolve quickly in saliva and the therapeutic agent retains activity for longer periods of time than prior art compositions and methods of applying them.

The present invention further relates to a dental composition having a therapeutic agent dispersed in a water-resistant carrier including a water-soluble fluid, solid or semi-solid, combined with a water-insoluble additive.

The present invention further relates to a solid or semi-solid therapeutic dental composition in a physical form that may be directly applied to an oral cavity surface, such as a dental crayon, thus obviating the need for a dental tray or other such delivery device.

The present invention still further relates to a therapeutic dental composition in packages that facilitate easy dispensing and application of said compositions into the oral cavity by a patient, dental practitioner, or consumer.

In another aspect of the present invention, a delivery device, such as a pen or brush, is used to dispense dental therapeutic compositions, such as tooth whiteners, directly onto the tooth and/or gum surfaces by a consumer, patient, dentist, or dental practitioner.

In yet another aspect of the present invention, a kit comprises at least one therapeutic water-insoluble or water-resistant dental composition, at least one set of instructions, and a device or secondary composition that assists said therapeutic water-insoluble or water-resistant dental composition in its functional utility.

### BRIEF DESCRIPTION OF THE DRAWINGS

FIG. 1 depicts one embodiment of a delivery device of the present invention.

FIG. 2 depicts a felt tip pen that may be utilized as a device for administering the therapeutic dental composition of the present invention.

FIG. 3 depicts a brush pen that may be utilized as a device for administering the therapeutic dental composition of the present invention.

### DETAILED DESCRIPTION

A therapeutic dental composition is herein described which may be in the form of a stick or dispensed from a delivery device, which can be used to apply one or more therapeutic agents to the oral cavity. The dental composition will contain one or more therapeutic agents dispersed in a water-insoluble or water-resistant carrier, and the therapeutic agents will be released from the water-insoluble or water-resistant carrier over a period of time. The direction of agent release may be towards the oral cavity surface on which it is situated, towards the lumen of the oral cavity, away from the oral cavity surface on which it is situated, or both.

The dental composition of the invention is comprised of a solid or semi-solid carrier and at least one therapeutic agent dispersed throughout the carrier. The therapeutic agent may be dissolved in the carrier or simply dispersed homogeneously in the carrier as an insoluble suspended solid particulate. The therapeutic agent may also be emulsified with the carrier, creating separate and discrete solid carrier and therapeutic agent phases within the composition. The emulsion may be either an agent-in-carrier emulsion or a carrier-in-agent emulsion, analogous to a water-in-oil emulsion. The dental composition may also be a solid or semi-solid emulsion with additional phases to those described above.

The therapeutic agent is placed in close proximity to the tissue surface, that is, dispersed or dissolved in a film of the carrier deposited by contacting the inventive dental composition with said tissue surface. The composition of the carrier can be varied so as to provide the dental composition with a short, medium, or long residence time on the oral tissue surface. Solubility of the carrier composition in water (or in saliva) is a major predictor of the, residence time for the resultant film deposited on an oral cavity surface. Rapidly dissolving films will release therapeutic agents faster than slowly dissolving or water-insoluble films. It may be desirable, in some cases, to prolong release of the therapeutic agent from the film by using a less water-soluble carrier composition; in other cases, quick release of therapeutic agent may be preferred, and a highly water-soluble, but water-resistant, carrier composition is used. One preferred embodiment is a carrier composition with reduced water solubility that will produce films that release more of the therapeutic agent towards the tissue surface onto which the film and therapeutic agent are applied.

The dental crayon composition is in a solid or semi-solid form at room temperature, defined herein as between about 20 and 30 degrees Celsius (C.). Immediately upon contact with the oral cavity tissue surface to be treated, the dental composition forms a film, which is also in a solid or semi-solid form. Moisture in the oral cavity may dissolve the film in a short or long time frame, thereby changing the film from a solid or semi-solid to a liquid (i.e. a dissolved or dispersed solid). In any event, the temperature of the oral cavity and its tissue surfaces (between about 30 and 39 degrees C.) should not be sufficient to melt the dental crayon composition when deposited as a film. Therefore, the dental crayon compositions of this invention are preferably solid or semi-solid at temperatures between 20 and 40 degrees C. In some cases, it may be desirable for the dental crayon composition to be a solid or semi-solid at any temperature up to about 85 degrees C., which is nearing the maximum tolerable temperature for humans drinking hot potable liquids. Compositions with high melting temperatures will be more resistant to liquefaction if subjected to hot potable liquids while the film is in place in the oral cavity.

The present invention describes a solid or semi-solid oral therapeutic dental composition comprising: (1) a pharmaceutically acceptable, film-forming, water-insoluble or water-resistant solid or semi-solid carrier, (2) an oral or dental therapeutic agent that is dissolved, dispersed, or otherwise homogeneously distributed throughout said carrier for the purpose of treating a disease, symptom or condition when applied to at least one surface of the oral cavity; and (3) optionally, auxiliary ingredients. Two or more oral or dental therapeutic agents may be employed within the same composition in order to treat multiple diseases, symptoms or conditions.

The above composition may be applied to one or more surfaces in the oral cavity, such as the teeth, gums or tongue, to effect a therapeutic, curative or cosmetic effect on or around the surface contacted. The use of a water-insoluble or water-resistant carrier prevents, reduces or slows the solubilizing effect of saliva on the deposited composition, thus prolonging the film's contact time with the oral cavity surface. Once in contact with the surface (tooth, gingival tissue, etc.), the inventive composition is then activated by the moisture in saliva by solubilizing, mobilizing, releasing or otherwise activating the oral care therapeutic agent dispersed in the carrier. The activated therapeutic agent thus slowly migrates out of the film in the direction of the oral cavity surface, exerting the aforementioned therapeutic or cosmetic effect.

As limited erosion of the film occurs, longer contact times with the oral cavity surface can be achieved. Lower concentrations of the therapeutic agent are thus possible than are conceivable with films that erode more rapidly, as much of the therapeutic agent migrates away from the intended treatment area after being solubilized in saliva.

The solid or semi-solid carrier may contain any number of water-insoluble or water-resistant ingredients, including one or more of the following, either alone or in combination: hydrogenated vegetable oils, waxes, fatty acid esters of glycerol, fatty acid esters of polygycerol (including diglycerol esters and polyglycerol-3 esters), fatty acid esters of sugar alcohols (including sorbitan monostearate), fatty acid esters of polyethylene glycol, petrolatum, and other orally acceptable, water-insoluble or water-resistant, solid or semi-solid substances. A water-insoluble carrier will not dissolve in saliva but will eventually be removed from the teeth by mechanical erosion. A water-resistant carrier is resistant to removal by contact with an aqueous solution. A water-insoluble or water-resistant carrier releases all or a substantial portion of the therapeutic agent before being eroded. The therapeutic agent is typically water soluble and is released from the carrier when water permeates into the film. Ingredients, polyvinylprrylodine (PVP), for example, may be added to make the carrier more readily absorb water.

Another type of solid or semi-solid water-resistant carrier may be provided which contains a water-soluble fluid, solid or semi-solid, combined with a water-insoluble additive. One such composition comprises a water-soluble or partially water-soluble polyethylene glycol (PEG) fluid, paste or solid matrix that includes one or more additives for rendering it water-resistant. Such additives may include oils, waxes and polymers that possess limited water solubility, but are compatible, soluble, or otherwise dispersible in the PEG fluid, paste or solid. Thus, the combined carrier composition described possesses limited solubility, and has utility in the practice of the overall invention.

Certain carrier embodiments may contain a water-resistant additive comprising a mixture of a high molecular weight water-soluble anionic polymer, such as carboxypolymethylene (Carbopol, Noveon) or hydrolyzed or unhydrolyzed methyl vinyl ether/maleic anhydride copolymer (Gantrez, ISP), together with a di- or trivalent ion such as calcium, zinc, or aluminum. Such ions may be present in the formulation as inorganic salts (such as calcium phosphate or zinc oxide) or organic salts (such as aluminum oxalate, calcium lactacte or zinc lactate). Upon contact with moisture, the anionic polymer and the di- or trivalent ion become partially or completely solubilized, thereupon forming a water-resistant, crosslinked polymer structure. The resulting water-resistant structure further reduces the solubility of a solid or semi-solid carrier as described above, the result being increased resistance to erosion of the film when attached to an oral cavity surface, such as the teeth or gums. Alternatively, a composition comprising the high molecular weight water-soluble anionic polymer can be applied to the tooth or gum surfaces, and subsequently and sequentially contacted with a second composition comprising a di- or tri-valent ion as described above in order to achieve the same degree of insolubilization of the anionic polymer on the tooth or gum surface.

The use of a water-insoluble or water-resistant carrier further supports stability of the dental composition and therefore lends to a longer shelf-life than compositions not including these carriers. A preferred shelf life ranges from six months to five years.

The concentration of carrier in the composition may be about 10%, 15%, 20%, 25%, 30%, 35%, 40%, 45%, 50%, 55%, 60%, 65%, 70%, 75%, 80%, 85%, 90%, 95%, 96%, 97%, 98%, 99% weight to weight of the composition.

In one embodiment, the carrier is about 55.0% (w/w) to about 99.0% (w/w) of hydrogented vegetable oil. In another embodiment, the carrier is about 30.0% (w/w) to about 60.0% (w/w) of petrolatum.

Therapeutic agents useful when applied to the oral cavity include those known to be effective against tooth decay or caries, tartar or calculus, dental plaque, halitosis, tooth stains, gingivitis, periodontal disease, oral ulcers, and other diseases, afflictions or symptoms of the oral cavity. Therapeutic agents may include antimicrobial agents, tooth whitening agents, anti-inflammatory agents, tooth desensitizers, anticaries agents, tartar control agents, tooth and gum surface protectants, and tooth stain prevention agents, for example.

Suitable antimicrobial agents known or anticipated to have utility in the inventive compositions include compounds with inhibitory activity against microorganisms found in the oral cavity. Compounds such as triclosan, chlorhexidine salts (such as chlorhexidine digluconate), cetylpyridinium chloride and domiphen bromide are suitable antimicrobial agents useful in the present inventive compositions.

Suitable tooth whitening agents include one or more peroxide-containing compounds, or more broadly, oxidizing compounds. Such oxidizing compounds include alkali metal percarbonates (such as sodium percarbonate), carbamide peroxide, sodium perborate, potassium persulfate, calcium peroxide, zinc peroxide, chlorine dioxide, sodium chlorite, hydrogen peroxide complexes (such as a PVP-hydrogen peroxide complex) and hydrogen peroxide.

Suitable anticaries agents include but are not limited to a source of fluoride ion. Fluoride sources include sodium fluoride, potassium fluoride, calcium fluoride, stannous fluoride, stannous monofluorophosphate and sodium monofluorophosphate. These sources should release anywhere from about 25 to about 3500 ppm of fluoride ion. The anti-caries agent may be present in an amount from about 0.05% to about 3.0%, preferably about 0.2% to about 1.0% by weight of the dental composition.

Suitable tartar control agents include but are not limited to zinc salts (e.g. zinc citrate trihydrate) and agents containing phosphorous (e.g. sodium tripolyphosphate). Inorganic phosphorous tartar control agents may include any of the pyrophosphates such as disodium pyrophosphate, dipotassium pyrophosphate, tetrapotassium pyrophosphate, tetrasodium pyrophosphate and mixtures thereof. Organic phosphorous compounds that may serve as tartar control agents include polyphosphonates such as disodium ethane-1-hydroxy-1,1-diphosphonate (EHDP), methanediphosphonic acid, and 2-phosphonobutane-1,2,4-tricarboxylic acid. Amounts of the polyphosphate may range from about 0.5% to about 20.0%, preferably from about 1.0% to about 8.0%, optimally from about 1.2% to about 4.5% by weight of the dental composition. As an alternative to phosphates, zinc salts may be utilized as anti-tartar agents. Most preferred is zinc citrate trihydrate. Amounts of the zinc salt may range from about 0.5% to about 20%, preferably from about 1.0 to about 8.0%, optimally from about 2.0% to about 6.0% by weight of the dental composition.

The concentration of therapeutic agent in the dental composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 6.0%, 6.5%, 7.0%, 7.5%, 8.0%, 8.5%, 9.0%,9.5%, 10.0%, 10.5%,11.0%,11.5% , 12.0%, 12.5%, 13.0%, 13.5%, 14.0%, 14.5%, 15,0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0% 24.5%, 25%, 25.5%, 26.0%, 26.5%, 27.0%, 27.5%, 28.0%, 28.5%,29.0%,29.5%,30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%,41.5%, 42.0%, 42.5%, 43.0%, 43.5%, 44.0%, 44.5%, 45.0%, 45.5%, 46.0%, 46.5%, 47.0%, 47.5%, 48.0% 48.5%, 49.0%, 49.5%, 50% weight to weight of the composition.

In one embodiment, the tooth whitening agent includes from about 2% to about 50% by weight of sodium percarbonate in the composition; preferably, from about 10.0% to about 25.0% by weight of sodium percarbonate in the composition; more preferably, about 10.0%. 15.0%, or 25.0% by weight of sodium percarbonate in the composition. In another embodiment, the oxidizing compound includes about 2% to about 50% of hydrogen peroxide; preferably, from about 10.0% to about 25.0% by weight of hydrogen peroxide in the composition; more preferably, about 6.0% by weight of hydrogen peroxide in the composition

Auxiliary ingredients contemplated to be included in the compositions of the present invention include thickeners, secondary film-forming agents, flavorants, humectants, sweeteners, surface active agents, emulsifiers, pH adjusting agents, stabilizing agents, secondary therapeutic agents, opacifying agents, colorants, and other product modifying or enhancing components.

Suitable thickeners are well-known in the art. Suitable secondary film-forming agents include but are not limited to PVP. In one embodiment, PVP is present in the composition in an amount of about 5% (w/w).

Suitable flavorants include but are not limited to oils derived from plants and fruits such as citrus oils, fruit essences, mint, peppermint oil, spearmint oil, capsaicin, clove oil, oil of wintergreen, anise, sassafras, sage, eucalyptus, marjoram, cinnamon, lemon, orange, banana, cherry, apple, pineapple, grape, strawberry, blueberry, tutti frutti, methyl salicylate, Hagelin flavoring #640047, Hagelin flavouring #640057, Hagelin flavouring #671009, Hagelin flavoring #671010, and the like. Those skilled in the art will recognize that natural and artificial flavoring agents may be used independently or combined in any sensorially acceptable blend.

Suitable humectants include but are not limited to glycerin, sorbitol, xylitol, mannitol, lactitol, maltitol, and other sugar alcohols, polyethylene glycol, propylene glycol, and other edible polyhydric alcohols and mixtures thereof.

Suitable sweeteners include but are not limited to sucrose, lactose, dextrose, maltose, dextrin, dried inverted sugar, fructose, levulose, galactose, corn syrup and their solids, sorbitol, mannitol, xylitol, hydrogenated starch hydrolysates, maltitol, sucralose, aspartame, salts of acesulfatne, alitame, saccharin and its salts, cyclamic acid and its salts, glycyrrhizin, dihydrochalcones, thaumatin, monellin, stevia extract and the like.

Suitable surface active agents include but are not limited to sodium lauryl sulfate, condensates of sorbitan mono-oleate with from about 20 to 60 moles of ethylene oxide (e.g., “Tweens” a trademark of ICI United States, Inc.), condensates of ethylene oxide with propylene oxide and condensates of propylene glycol (“Pluronics” a trademark of BASF-Wyandotte Corp.).

Suitable pH adjusting agents include but are not limited to sodium hydroxide, potassium hydroxide, ammonium hydroxide, sodium carbonate, potassium carbonate, TRIS and triethanolamine.

Suitable stabilizing and/or chelating agents include but are not limited to EDTA and its salts, citric acid and its salts, gluconic acid and its salts, etidronic acid (Dequest 2010), alkali metal pyrophosphates and alkali metal polyphosphates.

Suitable opacifying agents include but are not limited to titanium dioxide and zinc oxide.

Suitable colorants include but are not limited to FD and C-type dyes and lakes, fruit and vegetable extracts, titanium dioxide, and the like, alone or in combination.

The concentration of each auxiliary ingredient in the dental composition may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0% weight to weight of the composition.

Additional carriers, therapeutic agents, and excipients useful in the invention are listed in Remington's, The Science and Practice of Pharmacy (2000); Lieberman et al., Pharmaceutical Dosage Forms (2d ed. 1989); Merck Index (13th Ed.).

The inventive compositions are preferably in the form of stick, solid or semi-solid, such as a dental crayon, or may be attached to or disposed in a delivery device, such as a backing, carrier film, dispensing tube, a dispensing pen, or other such mechanical means for transferring the composition from a container or package to an oral cavity surface in need of treatment. In either case, the dental composition may be placed in direct contact with the oral cavity surface in need of treatment, or alternatively, the dental composition may be first placed in or on the delivery device, such as a dental tray or strip, the delivery device then used to carry the therapeutic dental composition into the oral cavity and thus into contact with the oral cavity surface or surfaces in need of cosmetic or therapeutic treatment.

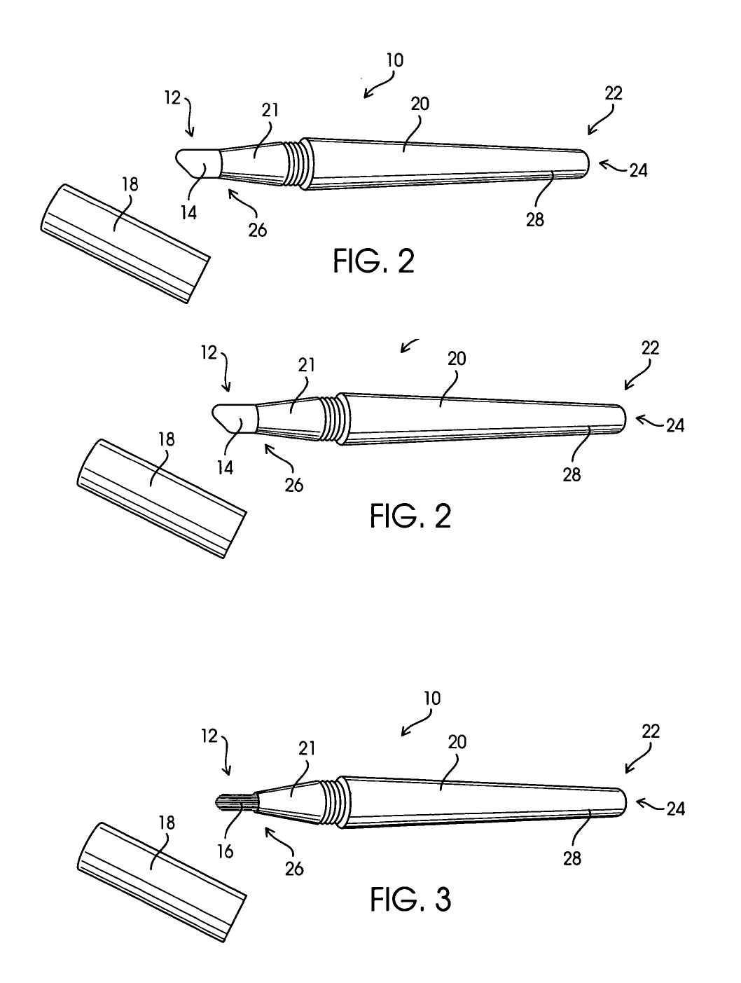

The inventive compositions are preferably disposed in a delivery device 10 (e.g., FIGS. 1-3), such as a dispensing tube, pencil, pen or crayon having an applicator 12, such as a felt tip 14 (FIG. 2), brush 16 (FIG. 3), roller ball, or non-woven pad. In one embodiment, the delivery device 10 includes more than one applicator 12 that may be removably engaged with the device 10. In an embodiment wherein the device 10 is a pen or a pencil, the applicator 12 may be retractable and/or housed in a cap 18. The therapeutic dental composition of the present invention may be housed directly within a reservoir 20 in the device 10 or may be supplied in a removable cartridge (not shown) within the reservoir 20 that may be replaced or refilled. The delivery device 10 may dispense the therapeutic dental composition through a transfer channel 21 through capillary action, such as in a flow through pen, or through an activator 22, such as mechanical piston with a click mechanism, twist button and ratchet mechanism, or push button mechanism, or through a vacuum method of ejection, or through other such mechanical means for transferring the composition from the device to an oral cavity surface in need of treatment. The activator 22 may be present on first end 24 of the device 10 and the applicator on a second end 26 of the device 10 or the activator 22 may be present on a side wall 28 of the device. In one embodiment, the delivery device 10 includes a felt tip 14 or brush 16 applicator 12 wherein the inventive composition is dispensed to the applicator 12 through actuation of the activator 22, such as by a clicking or twisting mechanism. Kotobuke Pencil, Japan, is one manufacturer of such types of delivery devices 10.

Preferably, the device 10 is free of metal components, more preferably made of plastic components. In one embodiment, the device is made from fluoropolymers, polypropylene, polyethylene, or other such polymers that are compatible with the ingredients of the composition of the present invention.

Upon applying external pressure to the activator 22 to expel the composition from the reservoir 20, the dental composition moves from the reservoir 20 through the transfer channel 21 to the applicator 12. Once the composition is positioned on the applicator 12, the user applies the composition to the teeth or gum surfaces, using the applicator 12 to apply and distribute the composition on the teeth and/or gums. Optionally, a set of instructions may be provided to the user in order that a particular application method or protocol be employed to apply the composition from the device 10 onto the teeth and/or gums in order to optimize the performance of the composition. With a twist mechanism, the user twists the activator 22 on the first end 24 of the delivery device 10 and the therapeutic dental composition travels from the reservoir 20 through the transfer channel 21 to the applicator 12 at the other end. With the push button activator 22, the therapeutic dental composition is delivered to the oral cavity surface with the push of a button activator 22 on the first end 24 or side wall 28, which transfers the composition from the reservoir 20 through the transfer channel 21 to the applicator 12.

The delivery devices 10 of the present invention may deliver a dose of the therapeutic dental composition upon each application to an oral cavity surface, for example, with each click or twist of the activator mechanism 22. The dose includes from about 0.01 mg to about 10 mg of therapeutic agent per dose, preferably from 0.1 mg to about 1.0 mg, more preferably from 0.1 mg to about 0.5 mg, and most preferably from 0.2 mg to about 0.3 mg of the therapeutic agent per dose. In one embodiment, the amount of dose dispensed from the device 10 may be adjusted by the user.

The pen delivery device 10 just described may also optionally comprise a set of bristles, advantageously positioned near or around the orifice through which the therapeutic dental composition is expelled. Expelling the therapeutic composition through the orifice and onto said bristles, the user may apply the therapeutic composition directly onto the teeth, thereby forming a film upon application. Alternatively, the dental composition may be brushed onto an oral cavity surface, using a brush (FIG. 2) or felt tip (FIG. 3) that is replenished with the therapeutic composition by returning it to a reservoir containing said composition or by clicking or twisting a dispensing portion of the reservoir. Yet another mode of application is placement of the inventive therapeutic composition into a dental tray, whereupon the dental tray is inserted into place around a patient's teeth. Plastic strips may also be coated with a predetermined dose of the therapeutic dental composition and placed against the teeth or gums of a subject. Alternatively, the inventive compositions may be applied by placing an amount on a swab or other such device, and simply applying directly to the intended oral cavity surface.

The dental stick or delivery device may be held in the hand and used by a patient in need of a cosmetic or therapeutic dental treatment, or by a separate individual, such as a dentist in an in-office procedure, to apply to the oral cavity of a patient. In the case of patient self-use, it is advantageous, but not required, for the patient to use the delivery device to apply therapeutic agents to the teeth, tongue and/or gums by using a mirror to guide placement and contact of the dental composition in the mouth.

A dental crayon is herein described as a solid or semi-solid object, usually in the form of a stick, which can be used to apply one or more therapeutic agents to the oral cavity. The stick can be formed into any shape or size that is aesthetically and functionally practicable. For example, a crayon may be formed having a length of about 6 cm, and a cross-sectional diameter of about 1 cm. The dental crayon provides a residual film on the tooth or gum surfaces. The residual film will contain one or more therapeutic agents, and the therapeutic agents will be released from the film over a period of time. The direction of agent release may be towards the oral cavity surface on which it is situated, towards the lumen of the oral cavity, away from the oral cavity surface on which it is situated, or both.

Methods of using the above compositions and devices are also contemplated. One such method involves identifying an oral cavity surface in need of treatment, applying the therapeutic dental composition described herein, and leaving said composition in contact with the oral cavity surface for a period of time sufficient to exert a therapeutic effect. Application of the composition in accordance with such a method may be performed, for instance, only once, or alternatively may be performed on a regularly scheduled basis, for instance one to three times a day for one week to four weeks, more preferably one to three weeks, most preferably for two weeks. Further, application of the composition may occur more than once a day for varying intervals of time. For example, the therapeutic dental composition may be administered to a subject one to six times per day, for a period of time ranging from 5 seconds to 8 hours per application, preferably 30 seconds to 2 hours per application, more preferably 2 minutes to 15 minutes, most preferably 15 minutes. In one embodiment, the composition is administered two times a day for fifteen minutes per application. In another embodiment, the composition is administered three times a day for fifteen minutes per application. It is also contemplated that the inventive compositions may be used on a daily basis, for instance, as a means of preventing tooth decay by including a fluoride ion-containing therapeutic agent in the composition. Sequential or concomitant application of two complementary, reactive or incompatible compositions is also contemplated, whereby at least one of the applied compositions possesses the inventive elements described above and disclosed elsewhere in this specification.

In one embodiment of the invention, the user first rinses the oral cavity with a mouth rinse prior to applying the dental composition of the present invention. The mouth rinse may be used to shift the pH of the oral cavity prior to administering the therapeutic dental composition, cleanse the oral cavity, or assist in making the therapeutic agent more adherent to the oral cavity surface to be treated.

The dental composition of the present invention may be used in a dental office setting or at home by the patient. In one embodiment, the dental composition is utilized in a light-activated procedure for tooth whitening or improving oral health. In a light-activated tooth whitening procedure, the dental composition may further include a photosensitzing agent or photosensitizer precursor as disclosed in U.S. Pat. No. 6,343,933.

In yet another embodiment, the therapeutic dental composition of the present invention may be used as a pre-treat or maintenance tool as a follow-up to in-office dental procedure, including but not limited to the procedures disclosed in U.S. Pat. No. 6,343,933 and PCT Publication No. WO 01/51005.

The present invention further contemplates kits comprising at least one therapeutic dental composition as described above, as a dental crayon or in a delivery device, and at least one set of instructions. In one embodiment, the fit further includes a secondary composition that assists the therapeutic dental composition in its functional utility.

It is believed that one skilled in the art, based on the description herein, can utilize the present invention to its fullest extent. The following specific examples are therefore to be construed as merely illustrative, and not limitative of the remainder of the disclosure in any way whatsoever.

### EXAMPLE 1

A tooth whitening stick (or crayon or pen) is prepared according to any one of the formulas of Tables 1 and 2.

TABLE 1

| | Formula (wt %) | | | | | | | | |
|---|---|---|---|---|---|---|---|---|---|
| Ingredient | 1 | 2 | 3 | 4 | 5 | 6 | 7 | 8 | 9 |
| Hydrogenated | 67.00 | 72.00 | 70.00 | 74.07 | 72.00 | 85.00 | 75.00 | 83.00 | 84.00 |
| Vegetable Oil | | | | | | | | | |
| Petrolatum | | | | | | | | | |
| Cetyl alcohol | | | | | | | | | |
| Beeswax | | | | | | | | | |
| Sodium percarbonate | 20.00 | 20.00 | 17.00 | 12.17 | 15.00 | 15.00 | 15.00 | 15.00 | 15.00 |
| powder | | | | | | | | | |
| Sodium bicarbonate | | | | | | | | | |
| powder | | | | | | | | | |
| Carbopol 974 | 10.00 | 5.00 | 10.00 | 10.58 | 10.00 | | 10.00 | | |
| Fumed Silica | 3.00 | 3.00 | 2.00 | 2.12 | 2.00 | | | 2.00 | |
| Flavor | | | 1.00 | 1.06 | 1.00 | | | | 1.00 |
| Total | 100.00 | 100.00 | 100.00 | 100.00 | 100.00 | 100.00 | 100.00 | 100.00 | 100.00 |

| | Formula (wt %) | | | | | | | |
|---|---|---|---|---|---|---|---|---|
| Ingredient | 10 | 11 | 12 | 13 | 14 | 15 | 16 | 17 |
| Hydrogenated | 88.00 | 89.00 | 97.00 | 67.20 | 58.80 | 69.00 | | |
| Vegetable Oil | | | | | | | | |
| Petrolatum | | | | | | | 60.00 | 60.00 |
| Cetyl alcohol | | | | | | | 5.00 | |
| Beeswax | | | | 16.80 | 25.20 | 15.00 | 10.00 | 12.00 |
| Sodium percarbonate | | | | 15.00 | 15.00 | 15.00 | 25.00 | 22.00 |
| powder | | | | | | | | |
| Sodium bicarbonate | | | | | | | | 5.00 |
| powder | | | | | | | | |
| Carbopol 974 | 10.00 | 10.00 | | | | | | |
| Fumed Silica | 2.00 | | 2.00 | | | | | 1.00 |
| Flavor | | 1.00 | 1.00 | 1.00 | 1.00 | 1.00 | | |
| Total | 100.00 | 100.00 | 100.00 | 100.00 | 100.00 | 100.00 | 100.00 | 100.00 |

TABLE 2

| | Formula (wt %) | | | | | | | |
|---|---|---|---|---|---|---|---|---|
| Ingredient | 18 | 19 | 20 | 21 | 22 | 23 | 24 | 25 |
| Petrolatum | 35.50 | 35.50 | 39.00 | 47.40 | 32.00 | | | |
| Paraffin Wax | 35.50 | 35.50 | 36.00 | 31.60 | 29.00 | | | |
| PEG 300 | | | | | | 10.00 | | |
| PEG 1000 | | | | | | | 50.00 | 70.00 |
| PEG 1450 | | | | | | 30.00 | | |
| PEG 3350 | | | | | | 50.00 | 24.00 | |
| PEG 8000 | | | | | | | | |
| Sodium percarbonate | 20.00 | 20.00 | 15.00 | 20.00 | 19.00 | | | |
| powder | | | | | | | | |
| Carbamide peroxide | | | | | | 16.00 | 16.00 | 22.00 |
| Citric acid | 5.00 | | | | | | | |
| Carbopol 974 | | 5.00 | 6.00 | | 5.00 | | 5.00 | |
| PVP | | | | | 12.00 | | | |
| Calcium lactate | | | | | | | 5.00 | |
| Zinc oxide | | | 1.00 | | | | | |
| Fumed Silica | 3.00 | 3.00 | 2.00 | | 2.70 | | | 2.00 |
| Flavor | 1.00 | 1.00 | 1.00 | 1.00 | 0.30 | | | 0.50 |
| Total | 100.00 | 100.00 | 100.00 | 100.00 | 100.00 | 100.00 | 100.00 | 100.00 |

The skilled artisan will appreciate that other therapeutic agents (e.g., hydrogen peroxide) can be added to and/or substituted for sodium percarbonate or carbamide peroxide in the above examples.

### EXAMPLE 2

The following compositions are applied to the oral cavity in a stepwise fashion, to effectuate tooth whitening.

Step 1 Composition (Mouthrinse)

| Ingredient | Percent (w/w) |
|---|---|
| Water | 89.400 |
| Potassium phosphate monobasic | 5.000 |
| Potassium phosphate dibasic | 5.000 |
| Cremaphor RH60 | 0.400 |
| Flavor | 0.200 |
| Total | 100.000 |

Step 2 Composition (Peroxide Stick)

| Ingredient | Percent (w/w) |
|---|---|
| Hydrogenated vegetable oil | 90.000 |
| Polyvinylpyrrolidone | 5.000 |
| Calcium peroxide | 5.000 |
| Total | 100.000 |

In this example, the user first rinses with 5-15 ml of the mouth rinse for about 30-240 seconds in step 1. Then, in step 2, the user rubs the above composition onto his/her teeth sufficient to thoroughly coat the teeth.

A number of embodiments of the invention have been described. Nevertheless, it will be understood that various modifications may be made without departing from the spirit and scope of the invention. Accordingly, other embodiments are within the scope of the following claims, and as various changes can be made to the above compositions, formulations, combinations, and methods without departing from the scope of the invention, it is intended that all matter contained in the above description be interpreted as illustrative and not in a limiting sense. All patent documents and references listed herein are incorporated by reference.

1-45. (canceled)

46. A tooth whitening composition, comprising:
about 50% to about 90% of a water-insoluble or water-resistant carrier; and
about 2% to about 50% of at least one whitening agent;
wherein percentages are weight to weight of the composition, and said composition is erodible in the oral cavity to allow the whitening agent to slowly migrate out from the carrier.

47. The tooth whitening composition of claim 46 wherein the water-insoluble or water-resistant carrier is selected from the group consisting of hydrogenated vegetable oil, wax, petrolatum, fatty acid ester of glycerol, a fatty acid ester of polyglycerol, a fatty acid ester of a sugar alcohol, and a fatty acid ester of polyethylene glycol.

48. The tooth whitening composition of claim 46 further comprising a film forming material.

49. The tooth whitening composition of claim 48 wherein said film forming material is polyvinypyrrolidone.

50. The composition of claim 49 wherein polyvinylpyrrolidone is present in the composition in an amount of about 12.0% (w/w).

51. The tooth whitening composition of claim 46 wherein said whitening agent comprises an alkali metal percarbonate, carbamide peroxide, sodium perborate, potassium persulfate, calcium peroxide, zinc peroxide, sodium chlorite, a hydrogen peroxide complex, hydrogen peroxide, or mixtures thereof.

52. The tooth whitening composition of claim 46 wherein said composition has a range of erosion times.

53. A method for whitening teeth of a subject comprising: providing a tooth whitening composition comprising:
about 50% to about 90% of a water-insoluble or water-resistant carrier; and
about 2% to about 50% of at least one whitening agent;
wherein percentages are weight to weight of the composition, and said composition is in a physical form for direct application to an oral cavity without the aid of a delivery device; and applying the composition to at least one tooth of the subject.

54. The method of claim 53 wherein said composition is erodible in the oral cavity to allow the whitening agent to slowly migrate out from the carrier.

55. The method of claim 53 further comprising a film forming agent.

56. The method of claim 53 wherein said whitening agent slowly migrates out of the composition onto the tooth.

57. The method of claim 53 wherein said water-insoluble or water-resistant carrier is selected from the group consisting of hydrogenated vegetable oil, wax, petrolatum, fatty acid ester of glycerol, a fatty acid ester of polyglycerol, a fatty acid ester of a sugar alcohol, and a fatty acid ester of polyethylene glycol.

58. The method of claim 53 further comprising the step of rinsing the oral cavity with a mouth rinse prior to application of the whitening composition to the tooth.

59. A method of using a therapeutic dental composition comprising:
applying to the surface of at least an area of an oral cavity a composition in physical form for direct application, comprising:
about 50% to about 90% of a semi-solid carrier comprising petrolatum; and
about 2% to about 50% of at least one therapeutic agent that is dissolved, dispersed, or otherwise homogeneously distributed throughout said carrier;
wherein percentages are weight to weight of the composition; and allowing the agent to slowly migrate out from the carrier.

60. The method of claim 59 wherein said carrier comprises a pharmaceutically acceptable, film-forming material.

61. The method of claim 59 wherein said dental composition is activated by the moisture in saliva by solubilizing, mobilizing, releasing or otherwise activating the therapeutic agent dispersed in the carrier.

62. The method of claim 59 wherein said therapeutic agent slowly migrates out of the carrier in the direction of the oral cavity surface.

63. The method of claim 60 wherein said erosion occurs when the composition is in contact with a surface of an affected area of the oral cavity.

64. The method of claim 59 wherein said therapeutic agent comprises antimicrobial agents, tooth whitening agents, anti-inflammatory agents, tooth desensitizers, anticaries agents, tartar control agents, tooth and gum surface protectants, or tooth stain prevention agents.

65. The method of claim 64 wherein said antimicrobial agents comprises triclosan, chlorhexidine salts, cetylpyridinium chloride or domiphen bromide.
